(19) United States
(12) Patent Application Publication
(10) Pub. No.: US 2026/0176695 A1
(43) Pub. Date: Jun. 25, 2026

(54) METHOD AND KITS FOR A TRANSCRIPTOMIC SIGNATURE WITH EMPIRICALLY DERIVED ALGORITHM CORRELATED TO THE PRESENCE OF ACUTE REJECTION ON KIDNEY BIOPSY IN TRANSPLANT RECIPIENT BLOOD

(71) Applicant: VERICI DX LIMITED, Penarth Cardiff West Glamorgan (GB)

(72) Inventors: Patricia CONNOLLY, Franklin, TN (US); Chan Ju WANG, London (GB); Nehal DOSHI, Wood Ridge, NJ (US)

(21) Appl. No.: 19/127,708

(22) PCT Filed: Nov. 1, 2023
(86) PCT No.: PCT/GB2023/052854

§ 371 Date: May 6, 2025

Related U.S. Application Data
(60) Provisional application No. 63/382,919, filed on Nov. 9, 2022.

(51) Int. Cl. C12Q 1/6883; G16B 25/10

(52) U.S. Cl. CPC C12Q 1/6883; G16B 25/10; C12Q 2600/118; C12Q 2600/158

(57) ABSTRACT

Disclosed herein is a method for a diagnostic tool to calculate a renal allograft recipient's risk for acute rejection.

### CROSS-REFERENCE TO RELATED APPLICATIONS

The present application claims the benefit of U.S. Provisional Patent Application No. 63/382,919, filed Nov. 9, 2022, which is incorporated herein by reference in its entirety.

### TECHNICAL FIELD

This disclosure relates to the field of molecular biology, and more particularly to detecting RNA transcriptomic molecular signatures. More particularly, this disclosure relates to methods for producing a risk score correlated to a renal allograft recipient's risk for acute rejection. The methods comprise analyzing the blood of renal allograft recipients by determining the expression levels of an RNA signature set comprising 17 preselected RNA transcripts with algorithm in order to identify acute rejection risk and monitor and guide treatment for such patients. A differential expression analysis can be applied to normalized expression read count (i.e. read counts of genes from next generation sequencing (NGS) technology) values of selected genes to derive a weighted cumulative risk score for the risk of acute rejection which can be calculated for each patient blood sample.

### BACKGROUND

Kidney transplantation is the treatment of choice for subjects with end stage kidney disease (ESKD) (Abecassis et al., Clin J Am Soc Nephrol 3:471-480, 2008). However, despite remarkable improvements in 1-year graft loss over the last decade, in each subsequent year after transplant, approximately 3% of kidney allograft recipients return to dialysis or require re-transplantation. Rates of late graft failure are relatively unchanged since the 1990s (Menon et al., J Am Soc Nephrol 28:735-747, 2017).

Indication of kidney allograft rejection relies mainly on monitoring methods such as proteinuria and serum creatinine. These measures may lead to assessment by biopsy. Currently, the diagnosis of clinical acute rejection requires a renal allograft biopsy which is most commonly triggered by an elevation of serum creatinine in the presence of renal injury, or, in the case of sub-clinical acute rejection, a renal allograft biopsy collected as part of surveillance protocols. Biomarkers which correlate to or predict the presence of acute rejection are needed to support clinical management in a sensitive and less invasive manner. Clinical acute rejection (AR), i.e. acute rejection associated with a decline in kidney function, occurs in approximately 10% of transplanted kidneys (Eikmans et al., Front Med 5:358, 2019). In addition, up to one-third of recipients have evidence of acute rejection on surveillance biopsy in the first 12 months despite not having a clinical decline in their kidney function (subclinical acute rejection) (Cippa, et al., Clin J Am Soc Nephrol 10:2213-2220, 2015; Nankivell et al., Am J Transplant 6:2006-2012, 2006; Rush et al., Clin J Am Soc Nephrol 1:138-143, 2006; and Zhang et al., JCI Insight 4(11), 2019). Chronic allograft damage, or interstitial fibrosis and tubular atrophy of unknown cause, account for most of the cases of graft loss. This has boosted research aimed at understanding and contrasting the mechanisms responsible for these late events, including, among others, alloantibody formation and recurrence of primary disease. Indeed, lack of long-term improvements despite dramatic reduction in the rate of acute rejection has challenged the assumption that acute rejection represents a major determinant of long-term graft outcomes. This assumption, however, contrasts with evidence that acute rejection with an inflammation/tubulitis score of 1 or above based on kidney biopsies, both clinically manifested and subclinical, negatively impacts long-term graft survival in patients receiving immunosuppressive regimens (Zhang et al., JCI Insight 4(11), 2019; Zhang et al., J Am Soc Nephrol 30(8): 1481-1494, 2019). Therefore, AR still represents one of the major targets for immunosuppressive therapy after transplantation. Data on the impact of subclinical rejection and borderline changes on graft outcomes are conflicting, and diagnosis is impacted by subjective reporting. Increasing evidence in the literature suggests that subclinical inflammation negatively impacts the allograft with the development of renal fibrosis and decline in renal function long term (Rampersad et al., Am J Transplant 22:761-771, 2022).

One of the main issues of current immunosuppressive protocols is the fact that they are not tailored to individual patient needs. Most patients receive a standardized immunosuppressive protocol resulting in some individuals being exposed to too much or too little immunosuppression with resultant complications. Early identification of individuals at highest or lowest risk of acute rejection could allow more targeted therapies aimed at improving long-term outcomes and reducing risk (Cippa, et al., Clin J Am Soc Nephrol 10:2213-2220, 2015).

This disclosure provides novel transcriptomic signature sets that can be used to identify risk of the presence of acute rejection episodes. The rigor of an NGS assay allows for the generation of performance characteristics, including accuracy and precisions, which better inform medical management of kidney transplant patients in a more personalized and predictive manner while accounting for full clinical continuum.

Tests for renal allograft rejection such as serum creatinine or proteinuria, may be insensitive and are late indicators of injury that elevate as warnings of rejection but are not entirely predictive. Such tests may include or lead to obtaining a biopsy specimen from the patient. There has been a need in the field for an improved test that does not require an invasive biopsy and is more predictive of the risk of allograft rejection.

### SUMMARY

In one aspect, provided herein is a method for identifying the risk that a renal allograft recipient is experiencing allograft rejection comprising the steps of: (a) isolating RNA from a biological specimen from the renal allograft recipient; (b) determining the expression levels of a preselected gene signature set in the specimen of the recipient; wherein the preselected gene set comprises the genes OSM, TSC22D1, ST8SIA1, RTN1, IFITM3, ANXA5, CAPS2, and SOCS3; (c) normalizing the expression levels of the preselected gene signature set; (d) calculating a risk score using an empirically derived algorithm from normalized expression levels of the preselected gene signature set; and (e) determining whether the recipient's risk score falls within high or low risk category for allograft rejection.

In some embodiments, the algorithm in the calculating step is a logistic regression model that utilizes the formula:

$$t = {log}_{b} \frac{p}{1 - p} = {β}_{0} + {β}_{1} {x}_{1} + {β}_{2} {x}_{2} + … + {β}_{M} {x}_{M} ,$$

wherein t is the risk score, β₀ is the y-intercept feature of the logistic regression algorithm, β₁ is the coefficient for a gene, and x₁ is the expression of the gene, to determine the probability of allograft rejection.

In some embodiments, the risk score varies between 0-100, and wherein a risk score of 51-100 indicates a high risk of experiencing allograft rejection. In some embodiments, the risk score varies between 0-100, and wherein a risk score of 0-50 indicates a low risk of experiencing allograft rejection.

In some embodiments, the preselected gene set comprises at least 9 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 10 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 11 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 12 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 In some embodiments, the preselected gene set comprises at least 13 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 14 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 15 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 16 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

In some embodiments, the expression levels are determined by a method selected from the group consisting of NanoString™, RNASeq NextSeq™, MiSEQ™ and quantitative polymerase chain reaction (qPCR).

In another aspect, provided herein is a method for selecting a renal allograft recipient for treatment to reduce the risk of renal allograft rejection which comprises (a) isolating RNA from a blood specimen from the renal allograft recipient; (b) determining the expression levels of a preselected gene signature set in the blood of the recipient; (c) normalizing the expression levels of the preselected gene signature set; (d) calculating a risk score using an empirically derived algorithm from normalized expression levels of the preselected gene signature set; (d) determining whether the recipient is at high risk or low risk for allograft rejection based on the risk score which is delivered to a clinician as an interpreted result; and (e) administering a treatment to prevent allograft rejection if the recipient is at high risk for allograft rejection,
wherein the preselected gene set comprises the genes OSM, TSC22D1, ST8SIA1, RTN1, IFITM3, ANXA5, CAPS2, and SOCS3.

In some embodiments, the calculating step is a logistic regression model that utilizes the formula:

$$t = {log}_{b} \frac{p}{1 - p} = {β}_{0} + {β}_{1} {x}_{1} + {β}_{2} {x}_{2} + … + {β}_{M} {x}_{M} ,$$

wherein t is the risk score, β₀ is the y-intercept feature of the logistic regression algorithm, β₁ is the coefficient for a gene, and x₁ is the expression of the gene, to determine the probability of allograft rejection.

In some embodiments, the risk score varies between 0-100, and wherein a risk score of 51-100 indicates a high risk of experiencing allograft rejection. In some embodiments, the risk score varies between 0-100, and wherein a risk score of 0-50 indicates a low risk of experiencing allograft rejection.

In some embodiments, the preselected gene set comprises at least 9 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 10 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 11 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 12 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 13 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 14 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 15 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises at least 16 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the preselected gene set comprises the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

In some embodiments, the expression levels are determined by a method selected from the group consisting of NanoString™, RNASeq NextSEQ™, MiSEQ™ and quantitative polymerase chain reaction (qPCR).

In some embodiments, the treatment to prevent allograft rejection comprises one or immunosuppressive therapies.

### BRIEF DESCRIPTION OF DRAWINGS

FIG. 1 shows a CONSORT Diagram of Study Enrollment for the clinical trial described in the Examples.

FIG. 2 shows a Process Diagram from Specimen Receipt Through Risk Score Generation. 1) The test is ordered, and blood specimen is collected and sent to lab. 2) RNA is isolated; cDNA library is prepared, and RNASeq is performed. 3) The sequencing data is uploaded and QC is assessed; files enter pipelines and proprietary algorithms that process data to produce test result. 4) Laboratory Director reviews assay and patient QC to approve release of results. Abbreviations: QC, quality control

FIGS. 3A and 3B show the clinical performance of the NGS 17-gene test vs Clinical Model. FIG. 3A shows clinical performance of the next generation sequencing test (solid line) was superior to the clinical model (creatinine at time of biopsy, dashed line) as demonstrated by the AUC and FIG. 3B shows that an applied threshold of 50 identified patients most likely to have a transplant rejection. Abbreviations: AR, acute rejection; AUC, area under the curve; Non-AR, non-acute rejection; NPV, negative predictive value; PPV, positive predictive value.

### DETAILED DESCRIPTION

### Definitions

In accordance with the present disclosure, there may be employed conventional molecular biology, proteomics, microbiology, recombinant DNA, immunology, cell biology and other related techniques within the skill of the art.

As used herein, the “expression level” of RNA disclosed herein generally means the mRNA expression level of the genes in the gene signature, or the measurable level of the genes in the gene signature measured in a sample, which can be determined by any suitable method known in the art, such as, but not limited to polymerase chain reaction (PCR), e.g., quantitative real-time PCR, “qRT-PCR”, RNA-seq, microarray, targeted gene expression sequencing (TRex), NanoString analysis, etc.

As used herein, “determining the level of expression,” “determining the expression level” or “detecting the level of expression”, as in, for example, “determining the expression level of a gene” refers to quantifying the amount of mRNA present in a sample and may or may not refer to normalized quantification. Detecting expression of the specific mRNAs can be achieved using any method known in the art as described herein. Typically, mRNA detection methods involve sequence specific detection, such as by RNASeq or qRT-PCR. mRNA specific primers and probes can be designed using nucleic acid sequences, which are known in the art.

“Control” or “non-rejection case” is defined as a sample obtained from a patient that received an allograft transplant who has had a biopsy which was interpreted to be negative for acute rejection, or “control” could be defined as a kit control in which standardized material, such as universal human reference RNA (UHR) is referenced.

As used herein, an “acute rejection” is defined as a rejection of a transplant (e.g., an allogenic renal transplant) that occurs early after the transplant, e.g., within 0-6 months after the transplant. In some embodiments, the early rejection occurs within 12 months of receiving the transplant. In some embodiments, the acute rejection is clinical. In some embodiments, the acute rejection is subclinical. In some embodiments, the acute rejection is T-cell-mediated. In some embodiments, the acute rejection is antibody-mediated. In some embodiments, the acute rejection is mediated by both T cells and antibodies. An acute rejection may be of any grade, including borderline.

### Gene Signatures

The gene expression profiles disclosed herein provide a blood-based assay and that is easily performed longitudinally on transplant patients. Renal transplant patients may be examined by their physician frequently post transplantation, with time intervals between visits gradually increasing over time. During these clinic visits, the patients' renal function and immunosuppression levels are usually monitored. The gene signatures described herein may be used to monitor the risk of a patient developing an acute rejection of a renal allograft. In some embodiment, the gene signatures described herein may be used to predict the risk of a patient developing an acute rejection of a renal allograft within about 30 days from the time of taking the clinical sample (e.g., a biopsy).

The present inventors have identified and validated a blood based 17-gene signature including algorithm in allograft recipients that produces a risk score which correlates to the presence or absence of acute rejection as identified histopathologically on kidney biopsy. Application of this gene set informs improvements in medical management of kidney transplant recipients in a more individualized manner with regard to immunosuppressive therapy.

The gene expression profile disclosed herein can be performed at the time of a routine monitoring clinical visit or in response to a clinical indication requiring further investigation. Demonstration of a positive test with no change in creatinine level would indicate subclinical inflammation and could lead to an increase in immunosuppression and/or discontinuation of the immunosuppression taper or decision to biopsy. Repeat testing, e.g., repeat testing using the gene signatures described herein, could guide the subsequent reduction in immunosuppression. For example, if two subsequent tests were low risk, the prednisone dose may decrease by 2.5 or 5 mg, or the target level for tacrolimus would be lowered by 0.5 mg/dl. If the test is high risk in the presence of an increase in creatinine, this would indicate a clinical acute rejection as evidenced by renal injury. In this instance, the patient would be treated with either high dose steroids or anti-lymphocyte agents depending on the overall immunological risk of the individual and the transplant centers' management procedures.

The risk score may be calculated using an empirically derived algorithm from normalized expression levels of the preselected gene signature set. The algorithm may be a logistic regression model that utilizes the formula:

$$t = {log}_{b} \frac{p}{1 - p} = {β}_{0} + {β}_{1} {x}_{1} + {β}_{2} {x}_{2} + … + {β}_{M} {x}_{M} ,$$

wherein t is the risk score, β₀ is the y-intercept feature of the logistic regression algorithm, and β₁ is the coefficient for the particular select gene as defined within the algorithm, and continues up, counting each gene in the signature; similarly, x₁ is the particular expression of that select gene in that patient at that time the blood was collected as determined by the test process. This continues up as well with x₂ being the expression level (normalized gene counts) of the next gene. in the signature and so forth until expression of all 17 genes with all 17 associated coefficients are included in the calculation of the result.

Gene expression may be normalized, for example, using variance stabilizing transformation (VST). VST is a methodology well know in the art and comprises the normalization of gene expression based on a fixed dispersion function. Zararsiz G, Goksuluk D, Korkmaz S, Eldem V, Zararsiz G E, Duru I P, Ozturk A. A comprehensive simulation study on classification of RNA-Seq data. PLOS One. 2017 Aug. 23; 12(8):e0182507. doi: 10.1371/journal.pone.0182507. PMID: 28832679; PMCID: PMC5568128.

Genes may be up or down regulated, and model coefficients β can be positively correlated to acute rejection or negatively correlated to AR.

The regression model generates probability scores between zero and one which are then converted (×100) to risk scores from zero to 100. The weighted cumulative score (r) can be used as a risk score for acute rejection for each patient. The risk score may then be categorically defined as low, intermediate or high risk based on defined cut-off points which are defined across the reporting range of 0 to 100 and for which there is a calculated predictive value of the risk for the patient experiencing an acute rejection. In some embodiments, a risk score of 51 or more indicates a high risk of the patient experiencing an acute rejection. In some embodiments, a risk score of 50 or less indicates a low risk of a patient experiencing an acute rejection.

The preselected gene signature set may comprise the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, DHCR24, or any combination or subset thereof. In some embodiments, the preselected gene signature set consists of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

### Methods of Use

In one aspect, the present disclosure provides a method for identifying a renal allograft recipient's risk of the presence of clinical or sub-clinical acute rejection and then at risk for graft loss comprising the steps of (a) isolating RNA from a blood specimen, (b) synthesizing cDNA from the RNA and using that to perform sequencing of the transcriptome, (c) determining the expression levels of each of the 17 genes in the gene signature set, (d) normalizing the expression counts from the 17 genes and utilizing them in a weighted manner using an empirically derived logistic regression algorithm to calculate a risk score; and (e) determining the interpretation as to whether the recipient is at high or low risk for the presence of acute rejection and then allograft loss. The genes in the gene signature set may be selected from the following genes: NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the genes in the gene signature are OSM, TSC22D1, ST8SIA1, RTN1, IFITM3, ANXA5, CAPS2, and SOCS3.

Pursuant to the present disclosure, a patient who has undergone renal transplant may have the assay of the present disclosure performed as part of their post-transplant follow-up and monitoring. The methods may comprise peripheral blood being taken, RNA being extracted, and RNA sequencing library of cDNA being generated. In some embodiments, this assay comprises performing RNA sequencing of the whole transcriptome, including some or all of the specific 17 signature genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the assay comprises sequencing the genes OSM, TSC22D1, ST8SIA1, RTN1, IFITM3, ANXA5, CAPS2, and SOCS3.

Expression levels may be determined for all or some of the 17 genes, and the acute rejection risk algorithm may be applied to determine the risk assessment score for individual patients. In some embodiments, if the patient's score is above a pre-defined cut-off point, the patient is categorized as being at high risk, in which case the patient may be evaluated to receive immunosuppression that will be managed in the same manner employed for a high risk patient e.g., immunosuppressive drugs such as calcineurin inhibitors (CNI's), avoidance of steroid withdrawal, avoidance of mTOR inhibitors (such as Sirolimus/Temsirolimus or Everolimus) or Belatacept. In some embodiments, if the score is below a pre-defined cut-off point, the patient is regarded as being at low risk for AR, in which case the patient may be a candidate for steroid withdrawal, or less aggressive regimens with mTOR inhibitors or Belatacept.

The exact cutoffs have been determined based on RNA sequencing carried out on the algorithm training cohort. Of note, the assay should be taken in the context of other clinical factors that would predefine a candidate as high or low risk e.g. age, serum creatinine and the presence of anti-HLA-antibodies, when planning the immunosuppression protocol for the patient. In some embodiments, a risk score is between 51 and 100 indicates a high risk of the patient experiencing an acute rejection. In some embodiments, a risk score of 0-50 indicates a low risk of a patient experiencing an acute rejection.

In one aspect, the present disclosure is directed to methods that accurately diagnose subclinical and clinical rejection and accurately identify allografts at risk for subsequent histological and functional decline and allograft recipients at risk for graft loss. When such high risk allograft recipients are identified, the present disclosure includes methods for treating such patients. The methods include, without limitation, increased administration of immunosuppressive drugs, i.e. a calcineurin inhibitor (CNI), such as cyclosporine or tacrolimus, or a less fibrogenic immunosuppressive drug such as mycophenolate mofetil (MMF) and/or sirolimus. The main class of immunosuppressants are the calcineurin inhibitors (CNIs). Steroids such as prednisone may also be administered to treat patients at risk for graft loss or functional decline. Antiproliferative agents such as Mycophenolate Mofetil, Mycophenolate Sodium and Azathioprine may also be useful in such treatments. Immunosuppression can be achieved with many different drugs, including steroids, targeted antibodies and CNIs such as tacrolimus.

The present disclosure is at least in part based on the identification of gene expression profiles expressed in a recipient of a kidney allograft transplant from living or deceased donors, that determine the risk for the probability of acute rejection as defined by histopathology phenotype on kidney biopsy. Without wishing to be bound by theory, it is hypothesized that the gene expression profile is predictive of subclinical as well as clinical acute rejection. This gives the clinician the ability to personalize the approach to the immunosuppression regimen, thereby maximizing immunosuppression in those at high risk and lowering immunosuppression in those with decreased risk.

For immunosuppression, an individual at lower risk (e.g., a patient with a risk score between 0 and 50) can, depending on other immunological factors, be treated with a reduced dose of MMF, a steroid-free regimen or with a “weaker” less frequently used primary immunosuppressant, such as Rapamycin, Sirolimus (Rapamune®), Everolimus (Zortress®) or Belatacept (Nulojix®). Utilizing this approach in lowering immunosuppression has been shown to reduce the risk of serious post-transplant infections and malignancies. These agents are recognized by those of ordinary skill in the art as less potent (or weaker) than other agents because they are associated with a higher risk of early acute rejection.

“Stronger” immunosuppressive agents include CNI's, such as tacrolimus (Prograf®, Advagraf®/Astagraf XL (Astellas Pharma Inc.), Envarsus XR® (Veloxis Pharma Inc.) and generics of Prograf® and cyclosporine (Neoral® and Sandimmune® (Novartis AG) and generics thereof. An individual at higher risk (e.g., a patient with a risk score between 51 and 100) may be treated with such stronger immunosuppressive agents.

In addition, if the gene expression profile identifies an individual as at risk for acute rejection (e.g., the patient has a risk score between 51 and 100), the patient may be subjected to more intensive monitoring of clinical laboratory results or gene expression profiles. In some embodiments, a patient is monitored using a method described herein once a month. In some embodiments, a patient is monitored using a method described herein every other month. In some embodiments, a patient is monitored using a method described herein every three months. In some embodiments, a patient is monitored using a method described herein every four months. In some embodiments, a patient is monitored using a method described herein every six months. In some embodiments, a patient is monitored using a method described herein once a year. In some embodiments, a patient is monitored using a method described herein twice a year. In some embodiments, a patient is monitored using a method described herein every two years.

In some embodiments, the present disclosure provides methods of calculating risk that a kidney allograft recipient is experiencing acute rejection comprising the steps of providing a blood specimen from a kidney allograft recipient, isolating RNA from the blood specimen, synthesizing cDNA from the mRNA, and measuring the expression levels of a 17 member gene signature set with algorithm present in the blood specimen. Non-limiting examples of methods of measuring expression levels include RNA-Seq, microarray, targeted RNA expression (TREx) sequencing (Illumina, Inc. San Diego California), NanoString (nCounter® mRNA Expression Assay-NanoString Technologies, Inc. Seattle Washington) or qRT-PCR. The results of the gene signature set analysis are compared to a pre-defined cut-off point. These methods are also described in Examples 1-6 below.

The 17 member gene signature set for use in practicing the methods disclosed herein may comprise the following genes: NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, DHCR24, and any combination or subgroup thereof. In some embodiments, the 17 member gene signature set for use in practicing the methods disclosed herein consists of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, 8, 9, 10, 11, 12, 13, 14, 15, 16 or 17 members of the 17 member gene signature set are analyzed in a method disclosed herein.

In some embodiments, any 8 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, and SOCS3 are analyzed.

In some embodiments, any 9 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and one of NCAPD2, KIF3B, STK24, PARN, DLG5, HNRNPAB, VDAC1, HLA-DPA1, and DHCR24 are analyzed.

In some embodiments, any 10 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. n some embodiments, the genes OSM, TSC22D1, ST8SIA1, RTN1, IFITM3, ANXA5, CAPS2, SOCS3, and two of NCAPD2, KIF3B, STK24, PARN, DLG5, HNRNPAB, VDAC1, HLA-DPA1, and DHCR24 are analyzed.

In some embodiments, any 11 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and three of NCAPD2, KIF3B, STK24, PARN, DLG5, HNRNPAB, VDAC1, HLA-DPA1, and DHCR24 are analyzed.

In some embodiments, any 12 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and four of NCAPD2, KIF3B, STK24, PARN, DLG5, HNRNPAB, VDAC1, HLA-DPA1, and DHCR24 are analyzed.

In some embodiments, any 13 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and five of NCAPD2, KIF3B, STK24, PARN, DLG5, HNRNPAB, VDAC1, HLA-DPA1, and DHCR24 are analyzed.

In some embodiments, any 14 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and six of NCAPD2, KIF3B, STK24, PARN, DLG5, HNRNPAB, VDAC1, HLA-DPA1, and DHCR24 are analyzed.

In some embodiments, any 15 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and seven of NCAPD2, KIF3B, STK24, PARN, DLG5, HNRNPAB, VDAC1, HLA-DPA1, and DHCR24 are analyzed.

In some embodiments, any 16 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein. In some embodiments, the genes OSM, TSC22D1, ST8SIA1, RTN1, IFITM3, ANXA5, CAPS2, SOCS3, and eight of NCAPD2, KIF3B, STK24, PARN, DLG5, HNRNPAB, VDAC1, HLA-DPA1, and DHCR24 are analyzed.

In some embodiments, each of the 17 genes of NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24 are analyzed in a method described herein.

In some embodiments of the methods disclosed herein, it is desirable to detect and quantify mRNAs present in a sample. Detection and quantification of RNA expression can be achieved by any one of a number of methods well known in the art. Using the known sequences for RNA, specific probes and primers can be designed for use in the detection methods described below as appropriate. Any one of NanoString, microarray, RNASeq, or quantitative Polymerase Chain Reactions (qPCR) such as Real Time Polymerase Chain Reactions (RT-PCR) or Targeted RNA sequencing (TREx) can be used in the methods disclosed herein. Nucleic acids, including RNA and specifically mRNA, can be isolated using any suitable technique known in the art. For example, phenol-based extraction is a common method for isolation of RNA. Phenol-based reagents contain a combination of denaturants and RNase inhibitors for cell and tissue disruption and subsequent separation of RNA from contaminants. In addition, extraction procedures such as those using TRIZOL™ or TRI REAGENT™, may be used to purify all RNAs, large and small, and are efficient methods for isolating total RNA from biological samples that contain mRNAs. Extraction procedures such as those using the QIAGEN-ALL prep kit and Promega Maxwell simplyRNA kit are also contemplated.

In some embodiments, use of quantitative RT-PCR is desirable. Quantitative RT-PCR is a modification of the polymerase chain reaction method used to rapidly measure the quantity of a nucleic acid. qRT-PCR is commonly used for the purpose of determining whether a genetic sequence is present in a sample, and if it is present, the number of copies or the relative quantity of copies compared to a reference sequence in the sample. Any method of PCR that can determine the expression of a nucleic acid molecule, including an mRNA, falls within the scope of the present disclosure. There are several variations of the qRT-PCR method that are well known to those of ordinary skill in the art. In some embodiments, the mRNA expression profile can be determined using an nCounter® analysis system (NanoString Technologies®, Seattle, WA). The nCounter® Analysis System from NanoString Technologies profiles hundreds of mRNAs, microRNAs, or DNA targets simultaneously with high sensitivity and precision. In this system, target molecules are detected digitally. The NanoString analysis system uses molecular “barcodes” and single-molecule imaging to detect and count hundreds of unique transcripts in a single reaction. The NanoString analysis protocol does not include any amplification steps.

In a typical embodiment, the central clinical laboratory will determine the expression values and calculate the risk score upon receipt of blood sample and requisition from an ordering clinician. The risk score along with interpretation will be returned to the ordering clinician who will evaluate the full clinical context for the patient, including the calculated acute rejection risk score and will utilize this information in medical management for the patient.

In an alternate embodiment, the assay will be performed as described above in a clinical laboratory but using a kit, and the results will be calculated through a web-based portal with access to the bioinformatic pipeline and algorithm and then returned electronically to the ordering clinician.

In a specific embodiment, provided herein is a method for identifying the risk that a renal allograft recipient is experiencing allograft rejection comprising the steps of:
(a) isolating RNA from a biological specimen (e.g., blood, tissue, or urine) from the renal allograft recipient;
(b) synthesizing cDNA from the RNA and sequencing the cDNA, then determining the expression levels of a preselected gene signature set in the specimen of the recipient; wherein the preselected gene set comprises at least the genes OSM, TSC22D1, ST8SIA1, RTN1, IFITM3, ANXA5, CAPS2, and SOCS3;
(c) normalizing the expression levels of the preselected gene signature set;
(d) calculating a risk score using an empirically derived algorithm from normalized expression levels of the preselected gene signature set; and
(e) determining whether the recipient's risk score falls within high or low risk category for allograft rejection based on the pre-defined cutpoints.

In some embodiments, the method further comprises step (f) reporting the subject's risk score. In some embodiments, the method further comprises step (g) determining whether to administer immunosuppressant treatment to the recipient.

The methods for method for identifying the risk that a renal allograft recipient is experiencing allograft rejection described herein may comprise analyzing more than the 8 genes OSM, TSC22D1, ST8SIA1, RTN1, IFITM3, ANXA5, CAPS2, and SOCS3. In some embodiments, the method comprises analyzing 12 genes (e.g., OSM, TSC22D1, ST8SIA1, RTN1, IFITM3, ANXA5, CAPS2, SOCS3, DLG5, HLA-DPA1, NCAPD2, and DHCR24). In some embodiments, the method comprises analyzing 12 genes selected from NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the method comprises analyzing 13 genes selected from NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the method comprises analyzing 14 genes selected from NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the method comprises analyzing 15 genes selected from NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the method comprises analyzing 16 genes selected from NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24. In some embodiments, the method comprises analyzing the 17 genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

The biological specimen (e.g., blood or a biopsy) to determine the expression level of the genes in the gene signature provided herein may be taken at an suitable time after transplantation. In some embodiments, the sample is taken one month, two months, three months, four months, five months, six months, eight months, nine months, ten months, 11 months, 12 months, 13 months, 14 months, 15 months, 16 months, 17 months, 18 months, two years, three years, four years, five years or longer after transplantation.

In some embodiments, repeat samples are taken for the purpose of monitoring the patient's risk of occurrence of acute rejection. In some embodiments, repeat samples are taken for the purpose of monitoring the patient's response to treatment. Monitoring the response to treatment may be used, for example, in cases where the patient has undergone treatment for a previously identified acute rejection, but for which the resolution of that rejection is not known without yet another biopsy. The methods provided herein may be used to determine whether the previously identified acute rection has likely resolved or not. If the patient continues to be at high risk of rejection, further treatment, or more aggressive treatment, may be warranted.

In some embodiments, a sample is taken once a month. In some embodiments, a sample is taken every other month. In some embodiments, a sample is taken every three months. In some embodiments, a sample is taken every four months. In some embodiments, a sample is taken every six months. In some embodiments, a sample is taken once a year. In some embodiments, a sample is taken twice a year. In some embodiments, a sample is taken every two years. Without wishing to be bound by theory, it is hypothesized that Tutivia can predict the risk of acute rejection occurring in the about 30 days prior to or following the biopsy.

In another aspect, provided here in are methods for identifying the risk that a renal allograft recipient is experiencing allograft rejection comprising the transcriptomic sequencing carried out in GOCAR as described in Example 6 below. In some embodiments, a first selection of candidate genes is obtained using weighted gene co-expression network analysis (WCGNA) (see, e.g., Langfelder et al., 2008. BMC Bioinformatics. 9, 599, which is incorporated herein by reference in its entirety) and differential gene expression analysis using DEseq2 (see, e.g., Love, et al., 2014 Genome Biol. 15, 550, which is incorporated herein by reference in its entirety) and supplemented with genes from a previous study (see Zhang et al., J Am Soc Nephrol 30(8):1481-1494, 2019, which is incorporated herein by reference in its entirety) based on the same cohort, as well as 263 genes identified in a meta-analysis across distinct cohorts. In such embodiments, boruta_py (see Kursa et al., 2010. J. Stat. Softw. 36, 1-13, which is incorporated herein by reference in its entirety), the Python implementation of the Boruta feature selection algorithm, may be used to further filter down the initial gene sets by selecting the genes most relevant to the outcome. In some embodiments, a logistic regression model is built using Optuna for hyperparameter optimization (see Akiba et al., 2019. Doi: 10.48550/arXiv.1907.10902, which is incorporated herein by reference in its entirety) with 5-fold cross validation throughout the parameter search.

A non-limiting example of the use of the gene signature set in predicting the risk for acute rejection is described below using the 17 gene signature set. In some embodiments, a method disclosed herein comprises the following four steps:
1) Training Set: A group of kidney transplant patients with known outcomes based on renal biopsy histopathology phenotypes will have a blood sample collected at or near the date of a for-cause or protocol kidney biopsy. The training set will have well-characterized associated data including demographics, related clinical data, medications and dosages, and histopathological results. The gene expression levels of the training set are used to derive the gene signature, including algorithm, for the test's risk score calculation.
2) Measuring the expression of the genes: Expression levels of the 17 genes from the blood samples renal transplant patients in the training set will be measured using any one of several well-known techniques. Use of the RNASeq, TREx, NanoString, microarray or qPCR techniques for measuring expression is described in Examples 2, 3 and 4 below. The expression level is represented differently based on the technology applied. For example, TREx uses the count of sequence reads that are mapped to the genes. qPCR uses CT (threshold cycle) values and NanoString uses the count of the transcripts.
3) Establishing an acute rejection risk score and cutoff: The differential expression analysis will be performed to compute the p values of gene features of desired expression level and read length. The p values reflect gene features of possible significance to the outcome of histopathology-defined acute rejection. Subset analysis and regularization are used to support the selection of the final gene set and to set the final risk score algorithm.
4) Once the final gest set and algorithm are defined by the training set, an independent validation set of kidney transplant patients are examined to determine the performance of that gene set and algorithm on an independent population. The validation set results are utilized to establish the effectiveness of the gene set+algorithm in clinical performance. Based on the risk score, the prediction statistics such as prediction AUC (area under the curve) of the ROC (Receiver operating characteristic) curve of the true positive rate versus the false positive rate at various threshold settings are obtained. ROC analysis can be used to determine the cutoff or optimal model and measure the overall prediction accuracy by calculation of the area under the curve, sensitivity/specificity, the positive predictive values (PPV) and the negative predictive values (NPV). An optimal risk score cutoff is established which best differentiates the high risk or low risk of acute rejection. It is expected that there will be a clear cutoff into two groups in that if a patient is in the high risk group they have a high likelihood of having acute rejection present on biopsy, and the test is interpreted to be positive. If a patient result is in the low risk group, they have a low likelihood of having acute rejection present on biopsy, and the test is interpreted to be negative.
5) Clinical Testing: In the clinical laboratory, the expression levels of the gene signature set for a new patient with unknown acute rejection risk are measured by the same technology used for the validation set. The risk score will be calculated and compared to the cut-point to determine the acute rejection risk score classification. The clinical laboratory will send the testing results to the ordering clinician.

Expression levels and/or reference expression levels may be stored in a suitable and secure data storage medium (e.g., a database). The database may interface with other appropriate and related systems such as a patient billing system, a laboratory freezer inventory system, or a laboratory information system.

“Recorded” refers to a process for storing information on computer readable medium, using any such methods known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present disclosure. A skilled artisan can readily appreciate that any number of the available computer-based system are suitable for use in the present disclosure. The data storage means may comprise any manufacture comprising a recording of the present information as described above, or a memory access means that can access such a manufacture.

### Kits

In another aspect, the present disclosure provides a kit for identifying renal allograft recipients who are at risk for acute rejection comprising in one or more separate containers primer pairs for the gene signature set: NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24, buffers, a housekeeping gene panel, primers for the housekeeping gene panel, positive control, negative control and instructions for use.

In certain embodiments, kits are provided for determining a renal allograft recipient's risk of the presence of acute rejection.

The kits may comprise primers for the 17 member gene signature set, optionally a housekeeping gene panel for TREx and NanoString assays (e.g., as described in Example 6), primers for housekeeping genes for qPCR assays and a control probe.

A kit can further comprise one or more RNA extraction reagents and/or reagents for cDNA synthesis. In other embodiments, the kit can comprise one or more containers into which the biological agents are placed and, preferably, suitably aliquoted. The kit may also contain printed instructions for use of the kit materials.

The components of the kits may be packaged either in aqueous media or in lyophilized form. The kits may also comprise one or more pharmaceutically acceptable excipients, diluents, and/or carriers. Non-limiting examples of pharmaceutically acceptable excipients, diluents, and/or carriers including RNAase-free water, distilled water, buffered water, physiological saline, PBS, reaction buffers, labeling buffers, washing buffers, and hybridization buffers.

The kits of the disclosure can take on a variety of forms. Typically, a kit will include reagents suitable for determining gene set expression levels (e.g., those disclosed herein) in a sample. Optionally, the kits may contain one or more control samples. In addition, the kits, in some cases, will include written information providing a reference (e.g., predetermined values), wherein a comparison between the gene expression levels in the subject and the reference (predetermined values) is indicative of a clinical status.

### EXAMPLES

The present invention is described further below in Examples which are intended to further describe the invention without limiting the scope thereof.

### Example 1: RNA Sequencing Assay: Identification of 17-Gene Set and its Application to Predict AR

### RNA Sequencing Assay Kit Includes:

1) Illumina TruSeq mRNA Library Prep Kit
2) TruSeq RNA Single Indexes Set
3) Promega Maxwell simplyRNA kit for extraction of high quality total RNA

### Methods for RNA Sequencing and Data Processing:

Total RNA was extracted from whole blood collected from kidney transplant recipients in the first 6 months following transplant using a Maxwell simplyRNA kit. Coding transcriptome cDNA libraries were generated with an Illumina TruSeq mRNA Library Prep Kit. The indexed libraries were sequenced on an Illumina NextSeq2000 or NextSeq550Dx sequencer. The reads with good quality were first trimmed, rRNA and HBB reads were filtered out, and remaining reads were aligned to human reference database. Resultant transcripts were then counted as the expression level normalized. These normalized count matrices were then used to calculate the acute rejection risk score using the 17 gene feature algorithm. Results passing QC were transformed to scale of 0-100 and reported as final acute rejection risk scores. The final acute rejection risk scores are further stratified into high or low acute rejection risk category based on pre-defined cut-off points.

### Example 2: Targeted RNA Expression (TREx) Assay

1) Custom Assay kit (primer sets for the 17 gene panel and a housekeeping gene panel (Example 6) and reagents)
2) Illumina® TruSeq® RNA Sample Preparation Kit v2
3) TruSeq RNA Single Indexes Set
4) QIAGEN RNeasy® Kit for extraction of high quality total RNA

### Targeted Expression TREx Experiments:

The total RNA will be extracted using the QIAGEN RNeasy® Kit. The sequencing library will be generated using the Illumina® TruSeq® RNA Sample Preparation Kit v2 by following the manufacturer's protocol: briefly, polyA-containing mRNA will be first purified and fragmented from the total RNA. The first-strand cDNA synthesis will be performed using random hexamer primers and reverse transcriptase followed by the second strand cDNA synthesis. After the end-repair process, which converts the overhangs into blunt ends of cDNAs, multiple indexing adapters will be added to the end of the double stranded cDNA. PCR will be performed to enrich the targets using the primer pairs specific for the gene panel and housekeeping genes. Finally, the indexed libraries will be validated, normalized and pooled for sequencing on the NextSeq2000 sequencer.

### TREx Data Processing:

The raw RNAseq data generated by the NextSeq sequencer (Illumina) will be processed using the following procedure: The reads with good quality will be first aligned to several human reference databases including the hg19 human genome, exon, splicing junction and contamination databases, including ribosomal and mitochondrial RNA sequences, using the BWA1 alignment algorithm. After filtering reads that mapped to the contamination database, the reads that are uniquely aligned with a maximum of 2 mismatches to the desired amplicon (i.e. PCR product from the paired primers) regions will be then counted as the expression level for the corresponding gene and further subjected to normalization based on the expression of the housekeeping genes.

### Example 3: NanoString Assay

1) Custom Codeset (barcoded probesets for the 17 gene panel, housekeeping gene panel (Example 6) and negative controls provided by NanoString).
2) nCounter® Master Kit including nCounter Cartridge, nCounter Plate Pack and nCounter Prep Pack.
3) QIAGEN RNeasy® Kit for extraction of high quality total RNA

### NanoString Experiments:

Total RNA will be extracted using the QIAGEN RNeasy® Kit by following the manufacturer's protocol; Barcode probes will be annealed to the total RNA in solution at 65° C. with the master kit. The capture probe will capture the target to be immobilized for data collection. After hybridization, the sample will be transferred to the nCounter Pre Station and the probe/target will be immobilized on the nCounter Cartridge. The probes are then counted by the nCounter Digital Analyzer.

mRNA Transcriptomic Data Analysis

The raw count data from the NanoString analyzer will be processed using the following procedure: the raw count data will be first normalized to the count of the housekeeping genes and the mRNAs with counts lower than the median plus 3 standard deviation of the counts of the negative controls will be filtered out. Due to data variation arising from the use of different reagent lots, the count for each mRNA from each different reagent lot will be calibrated by multiplying a factor of the ratio of the averaged counts of the samples on different reagent lots. The calibrated counts from different experimental batches will be further adjusted using the ComBat package.

### Example 4: qPCR Assay

1) Primer container (17 tubes with one qPCR assay per tube for each of the 17 genes, which include the 17 gene-panel and 2 housekeeping genes (ACTB and GAPDH) and the control probe (18S ribosomal RNA). The assays are obtained from Life Technologies.
2) TaqMan® Universal Master Mix II: reagents for qPCR reactions
3) TaqMan® ARRAY 96-WELL PLATE.
4) Agilent AffinityScript qPCR cDNA Synthesis Kit: for the highest efficiency of converting RNA to cDNA and fully optimized for real-time quantitative PCR (qPCR) applications.

Total RNA will be extracted from the allograft biopsy samples using the ALLprep kit (QIAGEN-ALLprep kit, Valencia, CA USA). cDNA will be synthesized using the AffinityScript RT kit with oligo dT primers (Agilent Inc. Santa Clara, CA). TaqMan qPCR assays for the 17-gene signature set, 2 housekeeping genes (ACTB, GAPDH) and 18S ribosomal RNA will be purchased from ABI Life Technology (Grand Island, NY). qPCR laboratory processes will be performed on cDNAs using the TaqMan® universal mix and PCR reactions will be monitored and acquired using a system. Samples will be measured in triplicate. Threshold cycle (CT) values for the prediction gene set as well as the 2 housekeeping genes will be generated. The ΔCT value of each gene will be computed by subtracting the average CT value for the housekeeping genes from the CT value of each gene.

### Example 5: RNA Transcriptomic Sequencing Assay Kit: Identification of 17-Gene Set and its Application to Predict AR

### RNA Sequencing Assay Kit Includes:

1) Illumina® RNA Prep with Enrichment, (L) Kit
2) IDT® for Illumina® RNA UD Indexes Set C, Ligation
3) Illumina NextSeq 1000/2000 P2 Reagents (200 Cycles)
3) QIAGEN PAXgene Blood RNA Kit (50)

### Methods for RNA Sequencing and Data Processing:

Total RNA was extracted from whole blood collected from kidney transplant recipients in the first 6 months following transplant using a QIAGEN PAXgene Blood RNA kit. Coding transcriptome cDNA libraries were generated with an Illumina® RNA Prep with Enrichment Kit. The indexed libraries were sequenced on an Illumina NextSeq2000 or NextSeq550Dx sequencer. The reads with good quality were first trimmed, rRNA and HBB reads were filtered out, and remaining reads were aligned to human reference database. Resultant transcripts were then counted as the expression level normalized. These normalized count matrices were then used to calculate the acute rejection risk score using the 17 gene feature algorithm. Results passing QC were transformed to scale of 0-100 and reported as final acute rejection risk scores. The final acute rejection risk scores are further stratified into high or low acute rejection risk category based on pre-defined cut-off points.

### Example 6: RNA Transcriptomic Sequencing Assay: Identification of 17-Gene Set and its Application to Predict AR

This example presents data from a non-randomized, prospective, observational international study (NCT04727788) to validate the ability of genomic tests to predict the risk of kidney clinical and subclinical AR, and chronic allograft damage.

### Methods

### Participants and Study Design

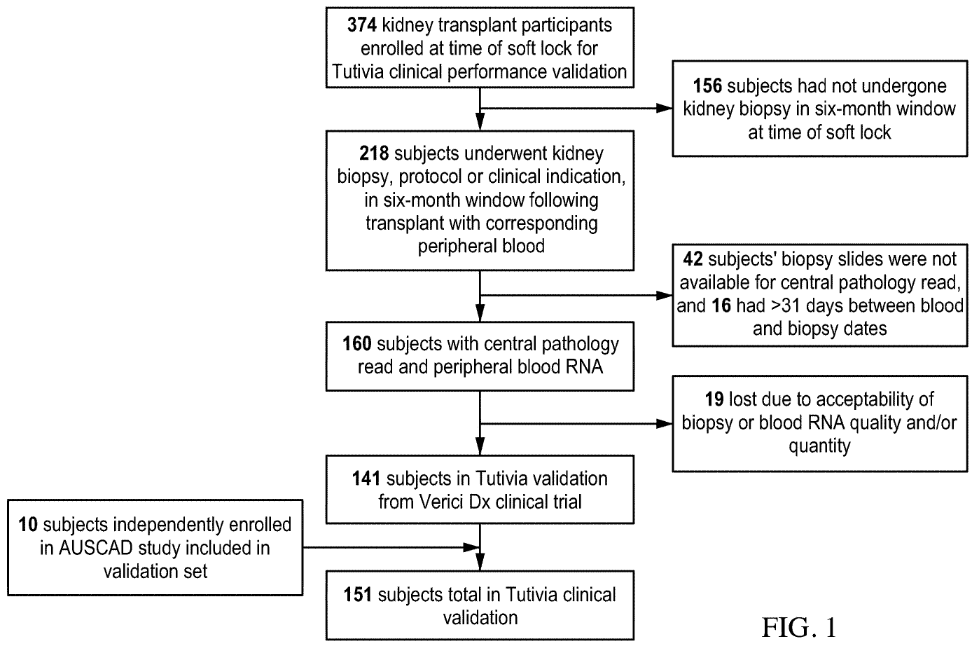

Thirteen study sites adhering to the Declaration of Helsinki were included in the validation set. Participants were enrolled into this study from March 2021 to January 2023. The study was approved by Advarra IRB, Pro00049177. Subjects from the Australian Chronic Allograft Dysfunction (AUSCAD) study were also included. Participants were included if they were a living or a deceased donor kidney transplant recipient, between 18 to ≤80 years of age and able to provide signed informed consent. A CONSORT diagram is provided in FIG. 1. Recipients of multiple organ transplant, excluding kidney-pancreas or patients who were participating in a therapeutic clinical trial for transplant rejection, with active HIV+ or Hepatitis C+, or pregnant were excluded. This observational study adhered to the Strengthening the Reporting of Observational Studies in Epidemiology (STROBE) reporting guidelines.

### Study Procedures and Specimen Collection

Study participants were evaluated at a pre-transplant visit where a detailed demographic, medical and transplant history was obtained including clinical characteristics of the donor. Following transplant, participants were asked to return at 1, 3, 6, 12 and 24 months post-transplant to provide medication updates and have laboratory, clinical and pathologic data collected. At 3 and 12 months, a core biopsy of the allograft was obtained either from a protocol mandated or standard surveillance procedure according to site protocol. Additionally, unscheduled visits for clinically indicated biopsies according to local site procedures were included. Blood samples were collected during all post-transplant visits. Peripheral blood was collected in 2 RNA PAXgene® tubes at all protocol and unscheduled visits. At the time of protocol biopsy visits, blood was collected within a median of 0 days from the correlate biopsy date. Twenty-three patients, in part due to COVID and related visit restrictions, along with site directed procedures requiring research blood-work to be obtained post-biopsy, had their blood taken within 31 days post-biopsy.

Given that the intent was to use the blood RNA signature to predict biopsy histology and risk of rejection, the timing of blood collection with a kidney biopsy is important for the current correlational study and subsequent clinical utility evaluation. Blood samples collected from allograft recipients at corresponding time points to biopsy were sent to the laboratory for testing.

All diagnostic kidney biopsies were first evaluated by the respective local site pathologist and then sent digitally (including all Hematoxylin and eosin-stained biopsies (H & E's) and any special stains along with C4D immunohistochemistry, when available) for a central pathology review. Approximately 5% of patients had glass slides sent for central pathology review. A secondary central pathology data review was independently obtained from approximately 15% of patients. The use of a secondary review was part of the initial study plan to adjudicate discrepant cases that were challenging, including borderline histology, C4D interpretation, or use of non-2019 BANFF criteria. Given the subjective semi-quantitative nature of histologic (immune cell/morphological) phenotyping, particularly for borderline classification, both local and central pathology diagnoses were evaluated. Therefore, when a discrepancy existed, there was a built-in allowance for an additional pathologist to derive a consensus (see Friedewald et al., Am J Transplant. 2019; 19(1):98-109). All biopsies were evaluated using 2019 BANFF criteria (Loupy et al. Am J Transplant. 2020; 20(9)).

HLA typing was performed according to individual local site and/or organ procurement organization protocols. Results of HLA typing are reported in the study and harmonized for assessment of number of relevant mismatches. H & E, periodic acid-Schiff (PAS), and C4d and SV40 immunohistochemistry for polyomavirus-associated nephropathy (PVAN) detection were examined via digital images or stained slides using standard diagnostic criteria for acute and chronic rejection, calcineurin inhibitor histopathologic features of toxicity, and other conditions that might affect the allograft. Acute cellular and antibody-mediated kidney rejection was determined using 2019 Banff criteria (see Friedewald 2019, surpa), while chronic damage was diagnosed as inflammation within areas of IFTA and scored by the chronic allograft damage index (CADI) and Banff 2019 guidance. Chronic active (CA) ABMR was defined according to BANFF system criteria (see Friedewald 2019, surpa). Study personnel, laboratory, central pathology and clinician investigators were blinded to results to reduce inherent bias.

### Primary Objective and Study Endpoints

The primary objective was to validate the prognostic performance of a peripheral blood gene expression signature (“Tutivia”) to predict risk of acute rejection through correlation with histopathology of surveillance or for-cause kidney biopsies. The primary outcome was evidence of clinical or subclinical rejection on histopathology of a kidney biopsy within 6-months post-transplant.

### Seventeen-Gene Signature Analysis

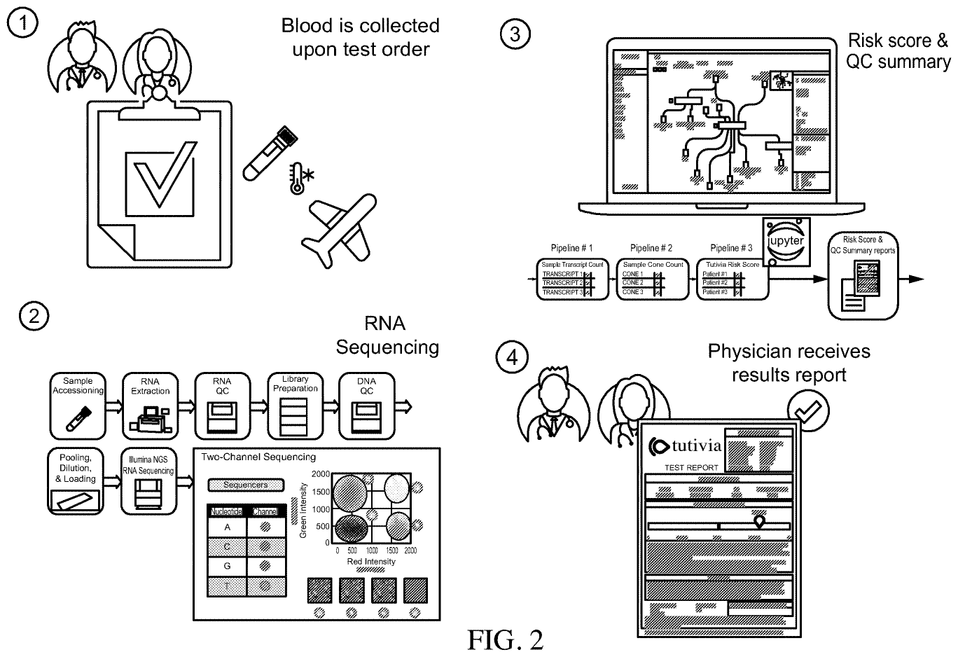

Total RNA was extracted from peripheral blood using a Promega Maxwell simplyRNA kit. Indexed transcriptome cDNA libraries were generated with an Illumina Stranded mRNA Library Prep Ligation Kit following the manufacturer's instructions. The indexed libraries were sequenced on an Illumina NextSeq2000. Good quality reads were first trimmed, with rRNA and HBB reads removed, before being aligned to the human reference genome database. Resultant counts were normalized prior to calculating the acute rejection risk score using the pre-defined 17-gene algorithm. All data processing was conducted using validated data processing and prediction pipelines. Results passing pre-specified quality control (QC) criteria were transformed to scale of 0-100 and reported as final acute rejection risk scores. The process from sample receipt to Tutivia risk score generation is detailed in FIG. 2.

The Tutivia algorithm incorporates quantitative measures of normalized individual gene transcripts, which are differentially weighted and assigned a value towards the computation of the final risk score. The final Tutivia 17-gene algorithm originated from the GoCar (see Zhang et al,. J Am Soc Nephrol. 2019; 30(8):1481-1494) cohort blood samples, which served as the training set. The GoCar samples were re-sequenced as defined in the Methods section above and the original findings were confirmed. Employing a novel, unbiased, unsupervised bioinformatic discovery interrogation process of >11,000 genes resulted in the current 17 gene signature. During this test development process only two genes from the original signature (Annexin A5 and TSC22D1) (see Zhang, 2019, supra) were identified, further establishing the uniqueness of the Tutivia gene set and algorithm. The complete list of the 17 genes in the Tutivia gene signature including ensemble ID/name, postulated role, and associated references are shown in Table 1. Each citation listed in Table 1 is incorporated herein by reference in its entirety.

TABLE 1
Genes in the RNA Signature.

| Gene ensemble | | Relevant |
|---|---|---|
| ID/Name | Known or postulated role(s) | References |
| ENSG00000099985 | This protein is a secreted cytokine and growth | Verstockt et al., |
| Oncostatin M | regulator. It also regulates the production of other | Inflamm Bowel |
| OSM | cytokines, including interleukin 6, granulocyte- | Dis. |
| | colony stimulating factor and granulocyte- | 2021; 27(10): 1564- |
| | macrophage colony stimulating factor in endothelial | 1575; West et al., |
| | cells. It is involved in positive regulation of acute | Nat Med. |
| | inflammatory response. | 2017; 23(5): 579-589 |
| ENSG00000101350 | The protein encoded by this gene acts as a | Lorenzen et al., |
| Kinesin Family | heterodimer with kinesin family member 3A to aid | Free Radic Biol |
| Member 3B | in chromosome movement during mitosis and | Med. 2013; 64: 78- |
| KIF3B | meiosis. It is involved in microtubule-based | 84; Duangtum et |
| | movement and the ATP catabolic process. KFI3B | al., Biochem |
| | plays an important role in trafficking kAE1 to the | Biophys Res |
| | plasma membrane. Impaired trafficking of kAE1 to | Commun. |
| | the basolateral membrane of α-intercalated cells of | 2011; 413(1): 69-74 |
| | the kidney-collecting duct causes distal renal tubular | |
| | acidosis (dRTA). | |
| ENSG00000102572 | This gene encodes a serine/threonine protein kinase | Jiang et al., Front |
| Serine/Threonine | that functions upstream of mitogen-activated protein | Immunol. |
| Kinase 24 | kinase (MAPK) signaling. The encoded protein | 2018; 9(APR) |
| STK24 | mediates oxidative-stress-induced cell death. STK24 | |
| | overexpression greatly enhanced IL-17-induced NF- | |
| | κB activation and expression of chemokines and | |
| | cytokines in a kinase activity-independent manner. | |
| ENSG00000102804 | This gene encodes a member of the TSC22 domain | Zhou et al., |
| TSC22 domain | family of leucine zipper transcription factors | Biochem Biophys |
| family, member 1 | involved in regulation of cell population | Res Commun. |
| TSC22D1 | proliferation and apoptosis. | 2018; 497(4): 1062- |
| | A single nucleotide polymorphism in the promoter | 1067; Jäger J et al., |
| | of this gene has been associated with diabetic | Mol Metab. |
| | nephropathy. TSC22D1 represents a regulatory | 2014; 3(2): 155-166 |
| | checkpoint for HDL-associated cholesterol levels | |
| | under both healthy and disease conditions. | |
| ENSG00000116133 | This gene encodes an oxidoreductase which | Spann et al., Cell. |
| 24- | catalyzes the reduction of sterol intermediates | 2012; 151(1): 138- |
| Dehydrocholesterol | during cholesterol biosynthesis. | 152; Körner et al., |
| Reductase | DHCR24 has been linked to Alzheimer's disease | Proc Natl Acad Sci. |
| DHCR24 | (AD), oncogenic and oxidative stress, hepatitis C | 2019; 116(41): 20623- |
| | virus (HCV) infections, differentiation of T helper- | 20634 |
| | 17 cells, development of foam cells, and prostate | |
| | cancer. This suggests a pronounced involvement of | |
| | DHCR24 in inflammatory processes, further | |
| | underscored by the actions of its substrate, | |
| | desmosterol. | |
| ENSG00000139970 | This gene belongs to the family of reticulon | Ling et al., Acta |
| Reticulon 1 | encoding genes. Reticulons are associated with the | Biochim Biophys |
| RTN1 | endoplasmic reticulum, which is an essential site for | Sin (Shanghai). |
| | the regulation of apoptotic pathways, and it has been | 2021; 53(2): 170- |
| | recently recognized as an important component of | 178; D' Eletto et |
| | autophagic signaling as well. | al., Cell Death Dis. |
| | | 2019; 10(12) |
| ENSG00000151208 | This gene encodes a member of the family of discs | Song et al., Biomed |
| Discs Large | large (DLG) homologs, a subset of the membrane- | Pharmacother. |
| MAGUK Scaffold | associated guanylate kinase (MAGUK) superfamily. | 2023; 162; Sezaki et |
| Protein 5 | The MAGUK proteins function as scaffolding | al., PLoS One. |
| DLG5 | molecules at sites of cell-cell contact. The protein | 2012; 7(4); Stoll M |
| | encoded by this gene localizes to the plasma | et al., Nat Genet. |
| | membrane and cytoplasm and interacts with | 2004; 36(5): 476-480 |
| | components of adherins junctions and the | |
| | cytoskeleton. It is proposed to function in the | |
| | transmission of extracellular signals to the | |
| | cytoskeleton and in the maintenance of epithelial | |
| | cell structure. | |
| ENSG00000164111 | The protein encoded by this gene belongs to the | Herrera-López et |
| Annexin A5 | annexin family of calcium-dependent phospholipid | al., Sci Rep. |
| ANXA5 | binding proteins, some of which have been | 2023; 13(1); Liu et |
| | implicated in membrane-related events along | al., Front Oncol. |
| | exocytotic and endocytotic pathways. Annexin 5 is a | 2023; 13 |
| | phospholipase A2 and protein kinase C inhibitory | |
| | protein with calcium channel activity with a | |
| | potential role in cellular signal transduction, | |
| | inflammation, growth and differentiation. It is | |
| | involved in negative regulation of the apoptotic | |
| | process. | |
| ENSG00000180881 | Calcyphosine-2 is a calcium-binding protein with 2 | Wang et |
| Calcyphosine 2 | EF-hand motifs. It is upregulated by cAMP and | al., Biochem |
| CAPS2 | thyrotropin in thyrocytes and can facilitate cell | Biophys Res |
| | proliferation. | Commun. |
| | | 2002; 291(2): 414- |
| | | 420; Shao et al., |
| | | Onco Targets Ther. |
| | | 2016; 9: 477-487 |
| ENSG00000184557 | This gene encodes a member of the suppressor of | Carow et al., Front |
| Suppressor Of | cytokine signaling (SOCS) family. SSI family | Immunol. |
| Cytokine Signaling | members are cytokine-inducible negative regulators | 2014; 5(FEB); Kubo |
| 3 | of cytokine signaling and are key physiological | et al., Nat |
| SOCS3 | regulators of the immune system. SOCS1 and | Immunol. |
| | SOCS3 regulate T cells as well as antigen- | 2003; 4(12): 1169- |
| | presenting cells, including macrophages and | 1176; Güler et al., |
| | dendritic cells. The expression of this gene is | Eur J Immunol. |
| | induced by various cytokines, including IL6, IL10, | 2020; 50(2): 234-244 |
| | and interferon (IFN)-gamma. This gene is | |
| | implicated in control of immune homeostasis in | |
| | physiological and pathological conditions such as | |
| | infection and autoimmunity. | |
| ENSG00000197451 | The hnRNPs are produced by RNA polymerase II | Lu et al.,. Cell |
| Heterogeneous | and influence pre-mRNA processing and other | death Discov. |
| Nuclear | aspects of mRNA metabolism and transport. | 2022; 8(1); Thibault |
| Ribonucleoprotein | Autoimmunity has an interesting role to play in A1 | et al., Biology |
| A/B | and A2/B1 mislocalization and aggregation. hnRNP | (Basel) |
| HNRNPAB | A/B family proteins have been recognized as | 2021; 10(8): 712 |
| | autoantibody targets in a subset of autoimmune | |
| | diseases, such as rheumatoid arthritis (RA), systemic | |
| | lupus erythematosus (SLE), mixed connective tissue | |
| | disease, scleroderma, Sjorgren's syndrome and MS. | |
| | Originally proposed as simple biomarkers of | |
| | disease, these antibodies are now hypothesized to | |
| | play a pathogenic role. | |
| ENSG00000213585 | This gene encodes a voltage-dependent anion | Hu et al., Cells. |
| Voltage Dependent | channel protein that facilitates the exchange of | 2022; 11(19); |
| Anion Channel 1 | metabolites and ions across the outer mitochondrial | Camara et al., Front |
| VDAC1 | membrane and may regulate mitochondrial | Physiol. |
| | functions. VDAC1 is the gatekeeper for the passages | 2017; 8(JUN) |
| | of metabolites, nucleotides, and ions. It plays a | |
| | crucial role in regulating apoptosis due to its | |
| | interaction with apoptotic and anti-apoptotic | |
| | proteins, namely members of the Bcl-2 family of | |
| | proteins and hexokinase. | |
| ENSG00000231389 | HLA-DPA1 plays a central role in the immune | Roche et al., Nat |
| Major | system by presenting peptides derived from | Rev Immunol. |
| Histocompatibility | extracellular proteins. Class II major | 2015; 15(4): 203-216 |
| Complex, Class II, | histocompatibility complex (MHC) molecules | |
| DP Alpha 1 | present antigens to CD4 positive cells. Class II | |
| HLA-DPA1 | molecules are expressed in antigen presenting cells | |
| | (APC: B lymphocytes, dendritic cells, macrophages) | |
| | and enable peptide antigen binding activity, | |
| | including antigen processing and presentation, | |
| | cellular response to type II interferon, and positive | |
| | regulation of type II interferon production. | |
| ENSG00000111728 | The protein encoded by this gene is a type II | Yang et al., Curr |
| ST8 Alpha-N- | membrane protein that catalyzes the transfer of sialic | Opin Endocrinol |
| Acetyl- | acid from CMP-sialic acid to GM3 to produce | Diabetes Obes. |
| Neuraminide | gangliosides GD3 and GT3. Ganglioside GD3 is | 2017; 24(2): 92-97; |
| Alpha-2,8- | known to be important for cell adhesion and growth. | Hu et al., Front |
| Sialyltransferase 1 | Evidence has demonstrated the implication of | Pharmacol. |
| ST8SIA1 | ST8Sia I and b- and c-series gangliosides in | 2022; 13. |
| | oncogenesis by mediating cell proliferation, | |
| | migration, tumor growth and angiogenesis. | |
| ENSG00000010292 | NCAPD2 enables histone binding activity, is | Yuan et al., World J |
| Non-SMC | involved in mitotic chromosome condensation, and | Gastroenterol. |
| Condensin I | plays pivotal role in chromosome assembly and | 2019; 25(47): 6813- |
| Complex Subunit | segregation during both mitosis and meiosis. | 6822; Zhang et al., |
| D2 | Overexpression of NCAPD2/3 promotes the release | Am J Pathol. |
| NCAPD2 | of pro-inflammatory cytokines by modulating the | 2020; 190(1): 37-47 |
| | IKK/NF-κB signaling pathway. | |
| ENSG00000140694 | Poly(A)-specific ribonuclease (PARN) plays an | Lata et al., Ann |
| Poly(A)-Specific | important role in regulating the stability and | Intern Med. |
| Ribonuclease | maturation of RNAs. Shortening of eukaryotic | 2018; 168(2): 100- |
| PARN | poly(A) tails is the rate-limiting step for mRNA | 109; Maragozidis et |
| | decay and translational silencing. PARN has been | al., Mol Cancer. |
| | found to regulate the maturation of the human | 2015; 14(1). |
| | telomerase RNA component (hTR), a noncoding | |
| | RNA required for telomere elongation. Mutations in | |
| | the PARN gene cause telomere diseases including | |
| | familial idiopathic pulmonary fibrosis (IPF) but how | |
| | PARN deficiency impairs telomere maintenance is | |
| | unclear. | |
| ENSG00000142089 | Interferon-induced transmembrane (IFITM) proteins | Guan et al., Kidney |
| Interferon Induced | are a family of interferon induced proteins involved | Int reports. |
| Transmembrane | in cytokine signaling within in the immune system | 2018; 3(4): 867-878; |
| Protein 3 | and are important innate immune effectors that | Yánez et al., |
| IFITM3 | prevent diverse virus infections. IFITM1-3 are | Immunology. |
| | expressed by T cells and recent experiments have | 2020; 159(4): 365- |
| | shown that the IFITM proteins are directly involved | 372 |
| | in adaptive immunity and that they regulate CD4+ T | |
| | helper cell differentiation in a T-cell-intrinsic | |
| | manner. | |

Of the 17 genes, 7 are associated with maintaining kidney function (including cellular division and metabolism), 4 are linked to immune pathways such as antigen processing, apoptosis and activation in T and B-cells, and 6 are part of various cytokine cascades that impact macrophage, neutrophil and NK cell activation and both antibody and T-cell based rejection and cell lysis. Some of these genes are directly related to high level dynamic processing of RNA and protein states within all cell types, which is directly related to immune pathways seen at the ‘static’ level of the biopsy. The final acute rejection risk scores were further stratified into high- or low-AR risk category based on a pre-defined score cut-off of 50.

### Statistical Analysis

The characteristics of study participants with and without rejection were compared using the Wilcoxon rank sum test (for continuous values) or the chi-square test (for categorical values). The ability of Tutivia to predict rejection was assessed by the area under the receiver operating characteristic curve (AUROC). The primary analysis was comparison of Tutivia versus a prespecified clinical benchmark model, i.e. creatinine at time of biopsy (see Gielis et al. Nephrol Dial Transplant. 2020; 35(4):714-721). The secondary analysis was the combination of the clinical model with Tutivia vs serum creatinine at biopsy with AUROC. It is noteworthy that the current benchmark model of serum creatinine at biopsy was not applied for subjects with specific adverse events (AE) (i.e., Delayed Graft Function, BK Viremia, Acute Kidney Injury and Acute Allograft Dysfunction) prior to for cause biopsies. The consensus was that these subjects did not have a reliable serum creatinine level (i.e., missing benchmark values) because of the impact of AE on the creatinine value. To reflect this, random values were imputed as the serum creatinine for these subjects to achieve a benchmark AUC of 0.5. All AUROC model measurements were calculated using bootstrapping, utilizing 500 replicates to correct for optimism. Given that AUROC reflects discrimination, all covariates were simply modeled as linear, and no variable selection procedures were performed. With the primary analysis being the comparison of two statistical prediction models, the study was powered using the methods developed previously (see Riley et al. Stat Med. 2019; 38(7):1276-1296). A sample size of 151 subjects provided 90% power to detect a 5% improvement in Nagelkerke's R-square for the Tutivia test over the benchmark model in predicting clinical and subclinical rejection.

The powering method described by Riley et al. was used. This method has been shown to be more accurate than the traditional 10 events per predictor rule (Riley et al., BMJ. 2020; 368). The online calculator for this calculation is available at https://riskcalc.org/samplesize/. All statistical analyses were conducted using R Studio, version 4.1.3 (R Foundation for Statistical Computing). A 2-sided p<0.05 was considered statistically significant.

### Results

### Validation Cohort Transcript Profile and Kidney Biopsy Characteristics

The current study is part of an ongoing global, non-randomized, observational trial for the validation of genomic tests to predict risk of kidney allograft clinical and subclinical AR. As identified in Table 2, there were 151 participants from 5 countries (US, France, Italy, Spain, and Australia). The median age of this cohort at time of transplant was 53 years, predominantly male (64%), and 79% were first time transplant patients. The mean time to acute rejection of biopsy was 57 days with an overall rejection rate of 31% (n=47). Race within the population was self-identified with 72% as White and nearly 21% as Black individuals (see Bureau, Racial and Ethnic Diversity in the United States: 2010 Census and 2020 Census. Accessed Dec. 9, 2022). The individual patient characteristics were also assessed for their significance in predicting risk of rejection. There were no restrictions on site immunosuppressive regimens (patients received ATG/Thymoglobulin with steroid (73 patients), interleukin 2 receptor subunit alpha (IL2RA) with steroid (49 patients), IL2RA without steroid (1 patient), Alemtuzumab with steroid (24 patients), steroid only (2 patients) or ATG/Thymoglobulin & IL2RA with steroid (2 patients)). The majority of the 151 patients (n=128, 85%) had their blood collected within one month prior to the day of biopsy and 15% (n=23 patients) had blood collected between 1 and 31 days post biopsy. Importantly, all 151 patients had received some form of induction therapy, including 48% (n=73) with ATG/Thymoglobulin with steroid, 33% (n=49) with IL2RA with steroid, and 19% (n=29) other combinations.

As noted in Table 2, the median donor age was 46 years, with 51 living donors and 100 deceased, of which 52 deceased donors were identified as standard criteria (SCD), 17 as expanded criteria (ECD), and 31 as donors following cardiac death (DCD). Four participants (2.6%) were blood type ABO incompatible, and 16 (11%) had positive (>30%) panel reactive antibodies (PRA) to both HLA class I and II at time of enrollment. Seventy-three patients (48%) had >4 HLA mismatches at A, B, DRB1, and DQB1.

### Patient Characteristics

The characteristics of patients in the 151 are listed in Table 2 below.

TABLE 2
Patient Characteristics

| | Total Cohort | | No Reject | | Reject | | |
|---|---|---|---|---|---|---|---|
| | N = 151 | | (N = 104) | | (N = 47) | | P-Value |
| Recipient Age in years, median | 53.0 | (18.0, 79.3) | 53.0 | (18.0, 79.3) | 52.0 | (22.0, 74.0) | 0.384 |
| (range) | | | | | | | |
| Recipient Sex, N (%) | | | | | | | 0.855 |
| Male | 97 | (64) | 66 | (64) | 31 | (66) | |
| Female | 54 | (36) | 38 | (37) | 16 | (34) | |
| Recipient Race, N (%) | | | | | | | <0.001 |
| Asian | 5 | (3) | 4 | (4) | 1 | (2) | |
| Black | 31 | (21) | 29 | (28) | 2 | (4) | |
| Native American | 0 | (0) | 0 | (0) | 0 | (0) | |
| Pacific Islander | 3 | (2) | 1 | (1) | 2 | (4) | |
| White | 108 | (72) | 67 | (64) | 41 | (87) | |
| Not Answered | 4 | (3) | 3 | (3) | 1 | (2) | |

Recipient Participation Location, N (%)

| USA | 84 | (56) | | | | | |
|---|---|---|---|---|---|---|---|
| Europe (Italy, France, Spain) | 57 | (38) | | | | | |
| Australia, | 10 | (7) | | | | | |
| Donor Age in years, median | 46.0 | (5.0, 81.0) | 45.0 | (5.0, 81.0) | 47.0 | (19.0, 79.0) | 0.214 |
| (range) | | | | | | | |
| Donor Sex, N (%) | | | | | | | 0.856 |
| Male | 76 | (50) | 52 | (50) | 24 | (51) | |
| Female | 62 | (41) | 41 | (39) | 21 | (45) | |
| Missing | 13 | (9) | 11 | (11) | 2 | (4) | |
| Donor Race, N (%) | | | | | | | 0.007 |
| Asian | 1 | (1) | 1 | (1) | 0 | (0) | |
| Black | 12 | (8) | 12 | (12) | 0 | (0) | |
| Native American | 0 | (0) | 0 | (0) | 0 | (0) | |
| Pacific Islander | 1 | (1) | 0 | | 1 | (2) | |
| White | 111 | (73) | 72 | (69) | 39 | (83) | |
| Not Answered | 26 | (17) | 19 | (18) | 7 | (15) | |
| PRA Class I, N (%) | | | | | | | 1.0 |
| 0% | 92 | (61) | | | | | |
| 1-30% | 19 | (13) | | | | | |
| >30% | 20 | (13) | | | | | |
| Not performed | 20 | (13) | | | | | |
| PRA Class II, N (%) | | | | | | | .615 |
| 0% | 93 | (62) | | | | | |
| 1-30% | 5 | (3) | | | | | |
| >30% | 33 | (22) | | | | | |
| Not performed | 20 | (13) | | | | | |
| Living Donor Recipient, N (%) | 51 | | | | | | 0.397 |
| Living Related Donor | 28 | | 22 | (21.2%) | 6 | (12.8%) | |
| Living Unrelated Donor | 23 | | 14 | (13.5%) | 9 | (19.1%) | |
| Deceased Donor Recipient, N (%) | 100 | | | | | | |
| Standard Criteria Donor | 52 | | 37 | (35.6%) | 15 | (31.9%) | |
| Expanded Criteria Donor | 17 | | 9 | (8.7%) | 8 | (17.0%) | |
| Donors after Cardiac Death | 31 | | 22 | (21.2%) | 9 | (19.1%) | |
| Previous Kidney Transplant Recipient, N (%) | | | | | | | 0.815 |
| 0 | 120 | (79) | 83 | (80) | 37 | (79) | |
| 1 | 18 | (12) | 11 | (11) | 7 | (15) | |
| 2 | 3 | (2) | 2 | (2) | 1 | (2) | |
| Missing | 10 | (7) | 8 | (8) | 2 | (4) | |
| ABO Incompatibility, N (%) | | | | | | | 0.589 |
| No | 147 | (97) | 102 | (98) | 45 | (96) | |
| Yes | 4 | (3) | 2 | (2) | 2 | (4) | |

| Cold Ischemia Time (CIT) for DD | N = 68 | | N = 32 | | 0.003 |
|---|---|---|---|---|---|
| Mean (SD) | 15.8 | (6.90) | 11.8 | (5.39) | |
| Median (Range) | 14.5 | (4.00, 39.9) | 9.00 | (3.00, 25.5) | |
| Missing, N (%) | 4 | (6) | 1 | (3) | |

| HLA Mis-matches (A, B, DRB1, DQB1), N | | | | 0.265 |
|---|---|---|---|---|
| 0-4 | 53 | 35 | 18 | |
| 5-8 | 82 | 55 | 27 | |
| Missing Data | 16 | 14 | 2 | |

Within 6 months post-transplant, all subjects had at least one surveillance or for-cause kidney biopsy which was histologically evaluated for rejection by a central pathologist using the BANFF 2019 criteria (Loupy et al. Am J Transplant. 2020; 20(9):2318-2331); 107 (71%) surveillance (protocol) and 44 (29%) for-cause (clinically indicated) biopsies were included. The comparison of central versus local pathology diagnosis is provided in Table 3.

TABLE 3
Biopsy pathology between local and central pathology diagnosis
(Central pathologist diagnosis is based on BANFF 2019 criteria)

| | | Central Biopsy Diagnosis | | |
|---|---|---|---|---|
| | | No Rejection | Rejection | Total |
| Local Biopsy | No Rejection | 101 | 23 | 124 |
| Diagnosis | Rejection | 3 | 24 | 27 |
| | Total | 104 | 47 | 151 |

As demonstrated in Table 3, the central pathologist classified approximately 50% more biopsies exhibiting evidence of rejection than the local pathologists. Given the subjectivity of the diagnostic process, utilizing a single expert pathologist to review all cases provides a level of diagnostic interpretive consistency, which is crucial for a correlative trial design as described in this report. Of the 47 allograft rejections, 20 (42%) were in the surveillance group with a median time to rejection of 97.5 days (78-133), and 27 (58%) in the for-cause group displayed a median time to rejection of 21 days (6-175). The median time to rejection for any biopsy was 58 days (6-175). Of the 47 AR, 11 were borderline TCMR, 13 TCMR-IA or higher, 12 ABMR, and 11 were classified as mixed rejections. Of the 23 patients whose blood was drawn after the biopsy, 18 (78%) were for-cause and 5 were surveillance biopsies. Of these, 8 classified as rejection by local pathology with 7 for-cause (4TCMR, 1 ABMR, 1 mixed) and 1 surveillance biopsy (mixed).

It is hypothesized that the 31% rejection rate was most likely the result of the addition of surveillance biopsies with for-cause biopsies and the inclusion of borderline within the rejection group. In support of this observation, a high rejection rate ranging from 29% to 46% in surveillance biopsies within 6 months of transplantation had previously been reported in several studies including one publication (Shapiro et al., Am J Transplant. 2001; 1(1):47-50) with a protocol biopsy at 8 days post-transplant (see Shapiro et al., Am J Transplant. 2001; 1(1):47-50; Nankivell et al., Am J Transplant. 2006; 6(9):2006-2012; Cippà et al., Clin J Am Soc Nephrol. 2015; 10(12):2213-2220; Zhang et al., JCI insight. 2019; 4(11); Crespo et al., Transplantation. 2017; 101(9):2102-2110).

### 3.2. Performance of the 17-Gene Tutivia Assay

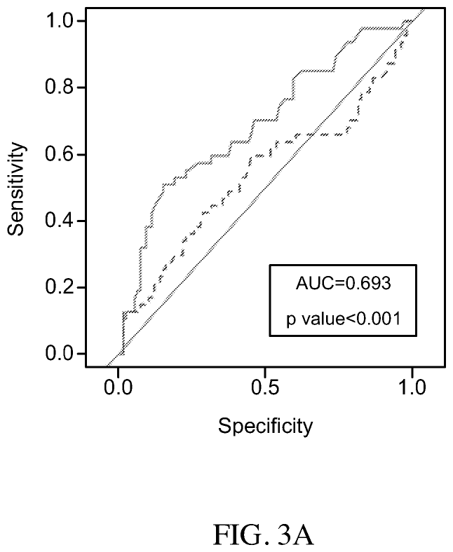

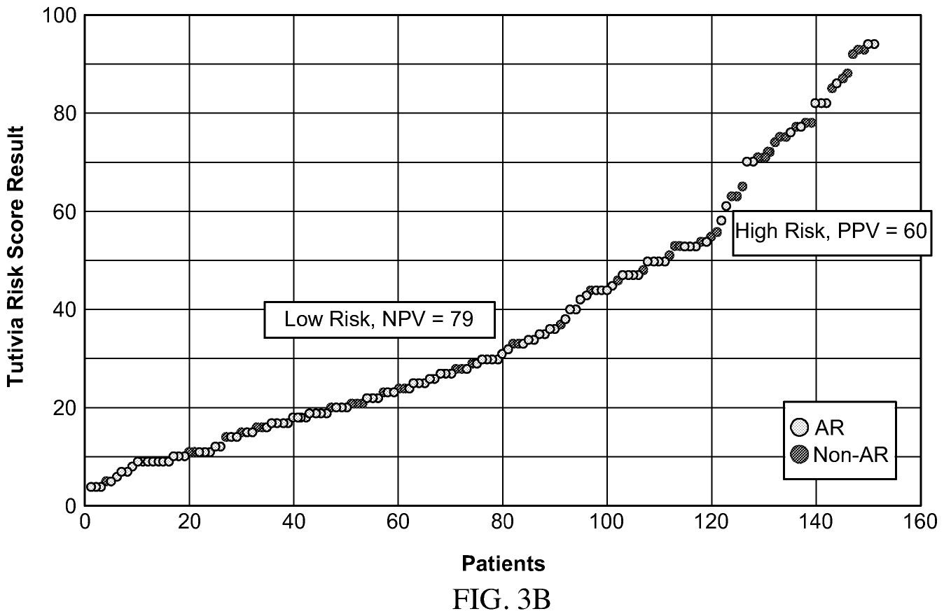

The 17-gene assay was evaluated using the receiver operating curve (ROC) with an AUC of 0.69 (95% CI 59.7-78.3) versus the baseline clinical model of creatinine at the time of biopsy with an AUC of 0.51 (95% CI 42.9-60.0), p-value=0.009. This demonstrated Tutivia as a continuous predictor for differentiating rejection from non-rejection (FIG. 3A). Also noteworthy is that even when combined with the baseline clinical model of creatinine at the time of biopsy (see Gielis et al., Nephrol Dial Transplant. 2020; 35(4):714-721) (AUC=0.68 (95% CI 59.2-77.2)) the 17-gene assay remained an independent predictor of transplant risk. Applying a predetermined cutoff of ≤50 as low risk and >50 as high-risk for rejection classified 40 patients as high-risk (26.5%) and 111 as low-risk (73.5%). Eighty-eight of the 111 low-risk patients had no AR, 7 had borderline AR, and 24 of the 40 high-risk patients with BANFF 2019 confirmed ARs, translated to an NPV of 79% and a PPV of 60% with an odds ratio of 5.74 (FIG. 3B and Table 4). Only 8 of the 23 patients whose blood was drawn after the biopsy (median 15 days) exhibited acute rejection by local pathology and 4 of the 8 (50%) had their blood taken within 10 days. The Tutivia assay correctly classified all 8 as rejection even though 7 out of the 8 patients had received some form of immunosuppressive therapy either on the day of biopsy or soon thereafter suggesting no significant impact (i.e no change in risk category) on the Tutivia signature.

TABLE 4
Performance of Tutivia with Model Cut-Off to Stratify Patients
into High- and Low-Risk Groups Utilizing Correlation to
Either a Surveillance or For-Cause Kidney Biopsy.

| Validation Model | Rejection | No Rejection | Total |
|---|---|---|---|
| Risk Score >50 | 24 | 16 | 40 |
| Risk Score ≤50 | 23 | 88 | 111 |

| Validation Performance Metric | Performance (95% CI) |
|---|---|
| Sensitivity | 0.51 (0.37-0.65) |
| Specificity | 0.85 (0.76-0.90) |
| PPV | 0.60 (0.45-0.74) |

| | | | |
|---|---|---|---|
| Validation Model | Rejection | No Rejection | Total |

| | |
|---|---|
| NPV | 0.79 (0.71-0.86) |
| Odds Ratio | 5.74 (2.63-12.54) |

PPV, positive predictive value;
NPV, negative predictive value

### 3.3. Clinical Subgroup Analyses

In the 151 patients, 35 (23%) clinically indicated (for cause) biopsies were performed prior to 60 days post-transplantation. Of these 35 early biopsies, 24 (69%) exhibited AR, and 20 (83%) had a high-risk Tutivia score suggesting a role for Tutivia as an early predictor of AR. Evaluation of Tutivia performance according to the type of clinical rejection identified in the for-cause biopsies to have a PPV of 0.75 (95% CI 0.57-0.87) and NPV of 0.63 (0.39-0.82) (Table 3). Table 5 provides additional performance metrics including sensitivity and specificity for both for-cause and surveillance biopsies (see Friedewald et al., Am J Transplant. 2019; 19(1):98-109; Bloom et al., J Am Soc Nephrol. 2017; 28(7):2221-2232; Halloran et al., Transplantation. 2022; 106(12):2435; Bixler and Kleiboeker, Donor-derived cell-free DNA: clinical applications for the diagnosis of rejection. Published online 2020; Lee et al., Semantic Scholar. Transplant. Published online 2023 and Oellerich et al., Am J Transplant. 2019; 19(11):3087-3099).

Although it is challenging to compare Tutivia with other commercially available tests predicting allograft rejection (due to assay type, trial design, BANFF endpoints and prevalence), the Tutivia PPV 0.75 and sensitivity of 0.78 were the highest for predicting for-cause rejection across all tests listed in Table 5. It is worth noting that the only other commercially available gene expression test listed in Table 5 is TruGraf (Friedewald et al., Am J Transplant. 2019; 19(1):98-109) that is contraindicated in the first 90 days. TruGraf was designed and validated for ruling out a need to biopsy in quiescent patients, which is quite different from Tutivia. Moreover, the current version of TruGraf has 120 genes in the algorithm and none overlap with Tutivia. This is not unexpected given TruGraf was developed using microarray techniques on surveillance only biopsies from quiescent kidneys with stable kidney function as a rule-out test. In contrast, Tutivia uses RNA sequencing and was developed as an ‘all-comers’ test regardless of a clinical state. Gene discovery in biomarker development is highly influenced by the design, training cohort, and clinical definition of rejection. For example, in TruGraf, a tubulitis score of t2 or t3 with i0 was classified as borderline (Park et al., Clin J Am Soc Nephrol. 2021; 16(10):1539-1551). This is divergent from BANFF criteria, whereas Tutivia is aligned to BANFF 2019. Meanwhile, in sub-clinical acute rejection (i.e. surveillance biopsy), Tutivia had a PPV of 0.25 (95% CI: 0.09-0.53), sensitivity 0.15 (0.05, 0.36) and a NPV of 0.82 (95% CI: 0.73-0.89), specificity 0.90 (0.81, 0.94). Additional efforts are underway to improve the prediction of rejection for Tutivia in the surveillance biopsy setting; however, in its current form the assay performs quite well for ruling out rejection in a sub-clinical setting. Similar challenges for other tests have also been reported (see Table 5) (Friedewald et al., Am J Transplant. 2019; 19(1):98-109); Halloran et al., Transplantation. 2022; 106(12):2435; Bixler & Kleiboeke, Donor-derived cell-free DNA: clinical applications for the diagnosis of rejection. Published online 2020; Lee et al., Transplant. Published online 2023; and Veríssimo Veronese et al., Clin Transplant. 2005; 19(4):518-521).

TABLE 5
Comparison of Tutivia vs. other commercially available tests predicting
allograft rejection in kidney transplant recipients

| | Tutivia | Allosure | Prospera | TruGraf | TRAC | VitaGraf |
|---|---|---|---|---|---|---|
| Test Type | 17 gene | dd cfDNA | dd cfDNA | 57 gene | dd cfDNA | dd cfDNA |
| | GEP RNASeq | | | GEP | | |
| | | | | microarray | | |
| Reference | | Bloom | Halloran | Friedewald | Bixler & | Oellerich |
| | | et al., J Am | et al., | et al., Am J | Kleiboeker, | et al., . Am J |
| | | Soc Nephrol. | Transplantation. | Transplant. | Donor- | Transplant. |
| | | 2017; 28(7): | 2022; 106(12): | 2019; 19(1): | derived | 2019; 19(11): |
| | | 2221-2232 | 2435 | 98-109 | cell-free | 3087-3099 |
| | | | | | DNA: | |
| | | | | | clinical | |
| | | | | | applications | |
| | | | | | for the | |
| | | | | | diagnosis of | |
| | | | | | rejection. | |
| | | | | | Published | |
| | | | | | online 2020. | |
| Primary | Multicenter, | Enrolled 1- | Multicenter, | Single | Post-hoc | Single |
| Validation | prospective, | 3 mo post tx | prospective, | center, 138 | 25 pts | center, |
| Study | 151 all-comer | or at time of | 218 patients | patients with | with 77 | prospective, |
| | patients at 13 | indication bx; | undergoing | only | samples, | 189 patients, |
| | sites enrolled | at 14 sites, | indication | surveillance | indication | 97 in test set |
| | pre-tx and | 107 samples | bx; | biopsy | bx, | (15 had AR) |
| | validated with | on 102 pts at | excludes | evaluating | 1 d-1146 d | |
| | indication or | indication bx | patients | quiescence | | |
| | surveillance bx | in val set | with BK | for tx | | |
| NPV | 79% | 84% | 83% | 78% | 86% | 98% |
| PPV | 75% in | 61% in | 71% in | 51% in | 55% in | 13% in |
| | indication | indication | indication | surveillance | indication | surveillance |
| | bx, 60% in | bx, excludes | bx, excludes | bx | bx, | bx |
| | indication bx + | borderline | borderline | | borderline | |
| | surveillance bx | AR | AR | | AR were | |
| | | | | | not analyzed | |
| | | | | | as AR | |
| Sensitivity | 78% in | 59% in | 74% in | 48% in | 58% in | 73% in |
| | indication bx, | indication bx, | indication bx, | surveillance | indication bx, | surveillance |
| | 51% in | excludes | excludes | bx | borderline | bx |
| | indication bx + | borderline | borderline | | AR were | |
| | surveillance bx | AR | AR | | not analyzed | |
| | includes | | | | as AR | |
| | borderline | | | | | |
| Specificity | 85% | 85% | 81% | 80% | 85% | 73% |
| AUC | 0.69 | 0.74 | 0.88 | 0.83 | 0.85 | 0.83 |
| | indication bx + | indication | indication | surveillance | indication | surveillance |
| | surveillance bx | bx | bx | bx | bx | bx |
| Prevalence | 31% | 25% | 39% | 30% | 20% AR, | 16% AR |
| | includes | excludes | excludes | subclinical | excludes | |
| | borderline | borderline | borderline | AR | borderline | |
| | AR | AR | AR | | AR | |

PPV, positive predictive value;
NPV, negative predictive value

Finally, kidney biopsies were evaluated for PVAN in those with SV40 staining; BK virus can be difficult to differentiate from rejection with current biomarker testing. 6 (4%) patient biopsies were identified as positive for PVAN. Compared to the group with negative SV40 (which included both rejection and non-rejection patients), SV40+ staining was highly correlated with low-risk Tutivia results (C=0.78).

### 4. Discussion

In this multicenter, international prospective study, the prognostic performance of Tutivia to predict risk of acute rejection was validated through correlation with histopathology of surveillance or clinically indicated kidney biopsies as determined by BANFF 2019 guidelines (see Nankivell et al., N Engl J Med. 2010; 363(15):1451-1462; Veríssimo Veronese et al., Clin Transplant. 2005; 19(4):518-521).

The results identified that 83% of the early indicated (clinical for cause) biopsies diagnosed with BANFF 2019 characterized rejection had a high-risk Tutivia score, highlighting a particular discrimination in predicting early clinical acute rejection with a PPV of 75% and an NPV of 63%. For surveillance biopsies Tutivia performed quite well for ruling-out rejection with an NPV of 82% and a specificity of 90%, compared to a suboptimal performance for identifying acute rejection with a PPV of 25% and sensitivity of 15%, as shown in Table 3. Furthermore, the generalized acute rejection prediction, with an NPV of 79% and a PPV of 60%, regardless of biopsy type, does support a broader clinical use of the Tutivia assay. This is especially important as the signature was validated in an all-comers, prospective, correlational real world evidence study, where clinically efficacious information is provided at both ends of the rejection spectrum.

The GoCAR study (see Zhang et al., J Am Soc Nephrol. 2019; 30(8):1481-1494) provided the initial feasibility evidence that peripheral blood transcripts can successfully identify individuals at higher risk of acute rejection and future graft loss at 3-months post-transplant. While serial surveillance biopsies could provide key information to characterize the current immune response and guide clinical care decisions, they are time consuming, costly, invasive, and often accompanied by increased risk of secondary complications. Thus, a non-invasive clinical bioassay that provides an assessment of the graft without the need for biopsy is highly advantageous (see Menon et al., J Am Soc Nephrol. 2017; 28(3):735-747; Eikmans et al., Front Med. 2019; 6(January):358; and Naesens et al., J Am Soc Nephrol. 2018; 29(1):24-34).

These data provide evidence that Tutivia is a useful assay to identify and potentially monitor both low- and high-risk kidney transplant recipients in various clinical scenarios. The current study design is prospective and inclusive of all-comer adult kidney transplant recipients with multiple site locations throughout the world, such that the results obtained are not biased by patient selection criteria or absence of diversity. Moreover, all study investigators and central pathologists were blinded to all study results to remove any bias in evaluating all patient kidney biopsies. Also unique to this study is that the majority of patients underwent a planned surveillance biopsy independent of suspected rejection, rather than only enrolling patients having a clinically indicated (for-cause) biopsy post-transplant.

A recent review article detailed the importance of noninvasive ‘liquid-biopsy’ approaches for predicting and monitoring transplant rejection, specifically for kidney transplant patients (Benincasa et al., Hum Immunol. 2023; 84(2):89-97). In this article the authors introduced the field of ‘transplantomics’, which emphasizes the necessity for a ‘network’ machine learning approach for deciphering and providing clarity when introducing ‘omics’ into clinical rejection. Tutivia has followed the machine learning strategy for gene identification and broad applicability to validate a generalizable signature that equates to a gene expression profile for predicting early AR. The diverse gene panel represents specific cell-based mechanisms of protein processing and receptor biology that are directly aligned with classical immune regulation supporting the complex ‘network’ referenced previously.

In addition, a recent completely independent post-hoc assessment of the Tutivia assay was published from a prospective randomized therapeutic trial on 21 patients to predict either subacute or clinical rejection (see Tawhari et al., Front Immunol. 2022; 13). The study reported a NPV of 0.92 (95% CI: 0.63-98.60) and a PPV of 0.70 (95% CI: 0.45-0.87) with an AUC of 0.83, further supporting the generalizability of the approach and promising performance of the Tutivia assay as a tool to predict the likelihood of rejection.

Serum creatinine has thus far been the most utilized test to assess kidney function and remains the gold standard in clinical practice as predictor of acute kidney injury (Aldea et al., Front Pediatr. 2022; 10:841). In the current study, and acting as a biomarker, Tutivia demonstrated significant improvement over the measure of serum creatinine in identifying acute kidney rejection. Moreover, in the event of monitoring and clinical need for surveillance biopsies, Tutivia was effective in ruling-out rejection and investigations are underway to ascertain the molecular drivers behind the diagnosed tissue-based (acute) rejection and relationship (if any) to long-term graft survival. Overall, Tutivia provided a more accurate prediction of acute rejection representing an improvement to current standard clinical care alone. Another important finding was the performance of Tutivia in 8 patient blood samples collected post-biopsy, 7 of which were for cause, and all had received different type and duration of treatment (except for the 1 subclinical biopsy patient). All were identified as high-risk by the Tutivia gene signature. Thus, the limited time window from biopsy to blood collection combined with the reported treatment variability supports the stability and robustness of the Tutivia gene signature in the acute setting.

Perhaps even more promising is the correlation of patients with BK nephropathy with lower Tutivia scores, allowing for the differentiation between acute graft rejection from viral related processes. Although clinically interesting and relevant to the field due to the absence of biomarkers to identify BK associated inflammation, additional patients with BK nephropathy are needed to further confirm these early observations.

### CONCLUSIONS

This study provides clinical validation of Tutivia as a noninvasive accurate predictor of (early) acute rejection beyond the current standard of care. Implementation of this blood-based transcriptomic signature assay offers a non-invasive baseline and future serial approach for clinicians to monitor a patient's wellbeing post-kidney transplant.

A number of embodiments of the invention have been described. Nevertheless, it will be understood that various modifications may be made without departing from the spirit and scope of the invention. Accordingly, other embodiments are within the scope of the following claims.

It is further to be understood that all values are approximate, and are provided for description. Patents, patent applications, publications, product descriptions, and protocols are cited throughout this application, the disclosures of which are incorporated herein by reference in their entireties for all purposes.

1. A method for identifying the risk that a renal allograft recipient is experiencing allograft rejection comprising the steps of:
(a) isolating RNA from a biological specimen from the renal allograft recipient;
(b) determining the expression levels of a preselected gene signature set in the specimen of the recipient; wherein the preselected gene set comprises the genes OSM, TSC22D1, ST8SIA1, RTN1, IFITM3, ANXA5, CAPS2, and SOCS3;
(c) normalizing the expression levels of the preselected gene signature set;
(d) calculating a risk score using an empirically derived algorithm from normalized expression levels of the preselected gene signature set; and
(e) determining whether the recipient's risk score falls within high or low risk category for allograft rejection.

2. The method of claim 1, wherein the algorithm in the calculating step is a logistic regression model that utilizes the formula:
$$t = {log}_{b} \frac{p}{1 - p} = {β}_{0} + {β}_{1} {x}_{1} + {β}_{2} {x}_{2} + … + {β}_{M} {x}_{M} ,$$
wherein t is the risk score, β₀ is the y-intercept feature of the logistic regression algorithm, β₁ is the coefficient for a gene, and x₁ is the expression of the gene,
to determine the probability of allograft rejection.

3. The method of claim 1 or 2, wherein the risk score varies between 0-100, and wherein a risk score of 51-100 indicates a high risk of experiencing allograft rejection.

4. The method of any one of claims 1-3, wherein the risk score varies between 0-100, and wherein a risk score of 0-50 indicates a low risk of experiencing allograft rejection.

5. The method of any one of claims 1-4, wherein the preselected gene set comprises at least 9 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

6. The method of any one of claims 1-4, wherein the preselected gene set comprises at least 10 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

7. The method of any one of claims 1-4, wherein the preselected gene set comprises at least 11 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

8. The method of any one of claims 1-4, wherein the preselected gene set comprises at least 12 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

9. The method of any one of claims 1-4, wherein the preselected gene set comprises at least 13 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

10. The method of any one of claims 1-4, wherein the preselected gene set comprises at least 14 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

11. The method of any one of claims 1-4, wherein the preselected gene set comprises at least 15 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

12. The method of any one of claims 1-4, wherein the preselected gene set comprises at least 16 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

13. The method of any one of claims 1-4, wherein the preselected gene set comprises the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

14. The method of any one of claims 1-13, wherein the expression levels are determined by a method selected from the group consisting of NanoString™, RNASeq NextSeq™, MiSEQ™ and quantitative polymerase chain reaction (qPCR).

15. A method for selecting a renal allograft recipient for treatment to reduce the risk of renal allograft rejection which comprises
(a) isolating RNA from a blood specimen from the renal allograft recipient;
(b) determining the expression levels of a preselected gene signature set in the blood of the recipient;
(c) normalizing the expression levels of the preselected gene signature set;
(d) calculating a risk score using an empirically derived algorithm from normalized expression levels of the preselected gene signature set;
(d) determining whether the recipient is at high risk or low risk for allograft rejection based on the risk score which is delivered to a clinician as an interpreted result; and
(e) administering a treatment to prevent allograft rejection if the recipient is at high risk for allograft rejection,
wherein the preselected gene set comprises the genes OSM, TSC22D1, ST8SIA1, RTN1, IFITM3, ANXA5, CAPS2, and SOCS3.

16. The method of claim 15, wherein the algorithm in the calculating step is a logistic regression model that utilizes the formula:
$$t = {log}_{b} \frac{p}{1 - p} = {β}_{0} + {β}_{1} {x}_{1} + {β}_{2} {x}_{2} + … + {β}_{M} {x}_{M} ,$$
wherein t is the risk score, β₀ is the y-intercept feature of the logistic regression algorithm, β₁ is the coefficient for a gene, and x₁ is the expression of the gene,
to determine the probability of allograft rejection.

17. The method of claim 15 or 16, wherein the risk score varies between 0-100, and wherein a risk score of 51-100 indicates a high risk of experiencing allograft rejection.

18. The method of any one of claims 15-17, wherein the risk score varies between 0-100, and wherein a risk score of 0-50 indicates a low risk of experiencing allograft rejection.

19. The method of any one of claims 15-17, wherein the preselected gene set comprises at least 9 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

20. The method of any one of claims 15-17, wherein the preselected gene set comprises at least 10 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

21. The method of any one of claims 15-17, wherein the preselected gene set comprises at least 11 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

22. The method of any one of claims 15-17, wherein the preselected gene set comprises at least 12 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

23. The method of any one of claims 15-17, wherein the preselected gene set comprises at least 13 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

24. The method of any one of claims 15-17, wherein the preselected gene set comprises at least 14 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

25. The method of any one of claims 15-17, wherein the preselected gene set comprises at least 15 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

26. The method of any one of claims 15-17, wherein the preselected gene set comprises at least 16 of the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

27. The method of any one of claims 15-17, wherein the preselected gene set comprises the genes NCAPD2, OSM, KIF3B, STK24, TSC22D1, ST8SIA1, RTN1, PARN, IFITM3, DLG5, ANXA5, CAPS2, SOCS3, HNRNPAB, VDAC1, HLA-DPA1, and DHCR24.

28. The method of any one of claims 15-27, wherein the expression levels are determined by a method selected from the group consisting of NanoString™, RNASeq NextSEQ™, MiSEQ™ and quantitative polymerase chain reaction (qPCR).

29. The method of any one of claims 15-28, wherein the treatment to prevent allograft rejection comprises one or immunosuppressive therapy.
